(19) United States
(12) Patent Application Publication
(10) Pub. No.: US 2023/0172529 A1
(43) Pub. Date: Jun. 8, 2023

(54) SYSTEM AND METHODS FOR FACILITATING NEUROMODULATION THERAPY BY AUTOMATICALLY CLASSIFYING ELECTROGRAPHIC RECORDS BASED ON LOCATION AND PATTERN OF ELECTROGRAPHIC SEIZURES

(71) Applicant: NeuroPace, Inc., Mountain View, CA (US)

(72) Inventors: Sharanya Arcot Desai, Mountain View, CA (US); Thomas K. Tcheng, Pleasant Hill, CA (US); Cairn G. Seale, Los Altos Hills, CA (US); Martha J. Morrell, Portola Valley, CA (US); David A. Greene, Fort Wayne, IN (US)

(21) Appl. No.: 18/070,902

(22) Filed: Nov. 29, 2022

Related U.S. Application Data
(60) Provisional application No. 63/285,416, filed on Dec. 2, 2021.

(51) Int. Cl. A61B 5/00; G16H 20/40

(52) U.S. Cl. CPC A61B 5/4094; A61B 5/7264; G16H 20/40; A61B 5/6868

(57) ABSTRACT

A method of assessing electrical activity of a brain includes, for each of a plurality of electrical-activity records of the brain, applying a machine-learned ESC model to the record to classify the record as one of a seizure record or a non-seizure record, wherein each of record is sensed by a corresponding one of a plurality of sensing channels of an implanted medical device; for each seizure record in a set of seizure records, applying the machine-learned ESC model to the seizure record to classify the seizure record as one of a local-seizure record or a spread-seizure record, wherein the seizure record comprises a first seizure record captured by a first sensing channel and a second seizure record captured by a second sensing channel; and for each spread-seizure record in a set of spread-seizure records, applying a machine-learned SSC model to the spread-seizure record to classify the spread-seizure record as a type of seizure spread pattern.

### CROSS-REFERENCE TO RELATED APPLICATION

This application claims the benefit of U.S. Provisional Application Ser. No. 63/285,416, entitled “System and Methods for Facilitating Neuromodulation Therapy by Automatically Classifying Electrographic Records Based on Location and Pattern of Electrographic Seizures” and filed on Dec. 2, 2021, which is expressly incorporated by reference herein in its entirety

### TECHNICAL FIELD

The present disclosure relates generally to systems and methods for classifying electrographic seizures, and more particularly, to systems and methods that classify electrographic seizures in terms of location and spread pattern through the application of machine-learned models to electrographic records.

### BACKGROUND

In epilepsy, manually physicians review patient data including electrographic records, e.g., EEG recordings, to determine possible treatment options for the patient. For a patients with an implanted neuromodulation systems, a manual review of EEG recordings sensed and stored by the neuromodulation system through implanted leads can inform treatment decisions for the patient. For example, a physician may review EEG recording to determine: 1) which implanted lead to deliver neuromodulation therapy through, 2) the stimulation parameters, e.g., frequency, pulse width, burst duration, amplitude, which define the neuromodulation therapy to be delivered, and 3) if the patient is a candidate for resective surgery or not.

In patients with bilateral seizure onsets, determining seizure laterality is of particular interest to physicians for making treatment decisions. Often treatment decisions are directly linked to seizure onset lead, activity type at the seizure onset, and seizure spread patterns. For example, if most of the seizure onsets are determined to be sensed by one particular lead, the physician may decide to program the neuromodulation system to only stimulate that lead. Similarly, if most of the seizure onsets are found to start with a particular type of activity, such as low voltage fast activity, the stimulation parameters of the neuromodulation system may be programmed to stimulate with high frequency stimulation, otherwise the neuromodulation system may be programmed with a different stimulation frequency.

Manually reviewing EEG recordings to determine seizure onset lead, activity type at the seizure onset, and seizure spread patterns can be very time-consuming, inefficient and prone to human error. Therefore, it is desirable to have a tool that automates the foregoing.

### SUMMARY

This disclosure relates to a method of assessing electrical activity of a brain. For each of a plurality of electrical-activity records of a brain, a machine-learned electrographic seizure classification (ESC) model is applied to the electrical-activity record to classify the electrical-activity record as one of a seizure record or a non-seizure record. Each of the plurality of electrical-activity records is sensed by a corresponding one of a plurality of sensing channels of an implanted medical device. For each seizure record in a set of seizure records, the (ESC) model is applied to the seizure record to classify the seizure record as one of a local-seizure record or a spread-seizure record. The seizure record comprises a first seizure record captured by a first channel of the plurality of sensing channels of the implanted medical device and a second seizure record captured by a second channel of the plurality of sensing channels of the implanted medical device. For each spread-seizure record in a set of spread-seizure records, a machine-learned seizure spread classification (SSC) model is applied to the spread-seizure record to classify the spread-seizure record as a type of seizure spread pattern.

An aspect of a treatment, e.g., stimulation site, stimulation parameters, etc., may be determined based on the type of seizure spread pattern for each spread-seizure record in the set of spread-seizure records. An aspect of treatment may also be determined based on the type of seizure spread pattern and a time of seizure spread for each spread-seizure record in the set of spread-seizure records. An aspect of treatment may be based on the type of seizure spread pattern, a seizure onset type for the first seizure record, and a seizure onset type for the second seizure record for each spread-seizure record in the set of spread-seizure records. In this case, a machine-learned activity type classification (ATC) model is applied to each of the first seizure record and the second seizure record to determine the seizure onset type for the first seizure record and the seizure onset type for the second seizure record.

This disclosure relates to an apparatus for assessing electrical activity of a brain. The apparatus includes a memory having one or more machine-learned models, including the ESC model, the SSC model, and the ATC model disclosed above. The apparatus also includes a processor that is coupled to the memory and configured to implement the above-disclosed method using the ESC model, the SSC model, and the ATC model. The apparatus may further include a treatment therapy suggestion module that is configured to process the information from the models, e.g., type of seizure spread pattern for each spread-seizure record in a set of spread-seizure records, type of seizure spread pattern and a time of seizure spread for each spread-seizure record in a set of spread-seizure records, and the type of seizure spread pattern, a seizure onset type for the first seizure record, and a seizure onset type for the second seizure record for each spread-seizure record in a set of spread-seizure records.

It is understood that other aspects of apparatuses and methods will become readily apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1 is a block diagram illustration of a system, including an implanted neurostimulation system, a records classification processor, and other external equipment, that enable classification of physiological records, e.g., EEG records, recorded by the neurostimulation system.

FIG. 2 is a perspective, schematic illustration of an implanted neurostimulation system implanted in a patient and configured to sense and record EEG records and provide such records as part of the system of FIG. 1.

FIG. 3 is a block diagram of the implanted neurostimulation system of FIG. 2, illustrating some of the functional subsystems of the system.

FIGS. 4A-4D are example visual representations of a physiological record in the form of an EEG record corresponding to electrical activity of the brain as recorded by an implanted neurostimulation system

FIGS. 5A-5I are illustrations of EEG records in chronological order as they may be displayed to a physician for manual review.

FIG. 6 is a block diagram illustration of a records classification processor of FIG. 1.

FIGS. 7A, 7B, and 7C are illustrations of EEG records after processing by the records classification processor of FIG. 6.

FIG. 8A is an illustration of a two-stage classification process implemented by a machine-learned electrographic seizure classification model and a seizure onset classification machine-learned model of the records classification processor of FIG. 6.

FIG. 8B are illustrations of three channel EEG records and their corresponding seizure classification output (seizure or non-seizure) of the machine-learned electrographic seizure classification model.

FIG. 8C is an illustrations of a four-channel EEG record and its corresponding seizure classification output (Lead1 and Lead2 seizure) of the machine-learned electrographic seizure classification model.

FIG. 9A is an illustration of a channel EEG record with a predicted time of seizure onset as determined by the records classification processor of FIG. 6.

FIG. 9B is an illustration of a four-channel EEG record and corresponding seizure onset times per channel, as predicted by the machine-learned electrographic seizure onset classification model.

FIG. 10 is a flow chart of a method of assessing electrical activity of the brain of a patient implemented by the records classification processor of FIG. 6.

FIGS. 11A-11C are flow charts of example processes implemented by the records classification processor of FIG. 6.

FIG. 12 is a schematic block diagram of an apparatus corresponding to the records classification processor of FIG. 1.

### DETAILED DESCRIPTION

Disclosed herein is an apparatus, e.g., a records classification processor, for assessing electrical activity of the brain of a patient having an implanted neurostimulation system that senses and records electrical activity through a number of sensing channels located at different areas of the brain. The apparatus includes a memory having a machine-learned electrographic seizure classification (ESC) model, a machine-learned seizure spread classification (SSC) model, a machine-learned activity type classifier (ATC) model, a treatment module, and a processor coupled to the memory.

The processor is configured to apply the machine-learned ESC model to each of a plurality of electrical-activity records of a brain, where each of the plurality of electrical-activity records is sensed by a corresponding one of a plurality of sensing channels. The ESC model is trained to classify a record as one of a seizure record or a non-seizure record. The ESC model is also trained or otherwise configured to further classify seizure records as either a spread-seizure record or a local-seizure record.

The processor is further configured to apply the machine-learned SSC model to a set of records classified by the ECS model as spread-seizure records to further classify the spread-seizure records as having a type of seizure spread pattern. The set of the seizure records comprises a first seizure record associated with a first channel of the plurality of sensing channels and a second seizure record associated with a second channel of the plurality of sensing channels. The SSC model is trained to predict a time of seizure onset for each seizure record in the set. The SSC model is also trained or otherwise configured to determine the seizure spread pattern based on the predicted times of seizure onset determined by the SSC model. The seizure spread pattern may be one where the seizure starts at a first channel located at a first area of the brain, e.g., at a first side of the brain, and expands or spreads to a second channel located at a second area of the brain, e.g., at a second side of the brain. The seizure spread pattern may be one where the seizure appears to start at about the same time at both of a first channel and a second channel.

The processor is further configured to apply the ATC model to records classified by the ECS model as seizure records—both local-seizure records and spread-seizure records. In one embodiment, the ATC model is applied only to the channel of a seizure record that has the earlier seizure onset. The ATC model is trained to determine the activity type, e.g., low voltage fast, hypersynchronous, rhythmic delta, etc., of these seizure records.

The treatment module is configured to determine an aspect of a treatment for the patient based on the various classifications determined by the ESC, SSC, and ATC models.

The records classification processor provides a tool that automatically classifies EEG records of a patient based on lead or channel of seizure onset, classifies the activity type seen at the onset, and determines the time of spread to other channels or leads. This tool replaces traditional manual review of EEG recordings and saves time and may lead to more accurate EEG classifications. The output of such an automated EEG classification tool can be used to automatically determine treatment options for patients.

Overview of System

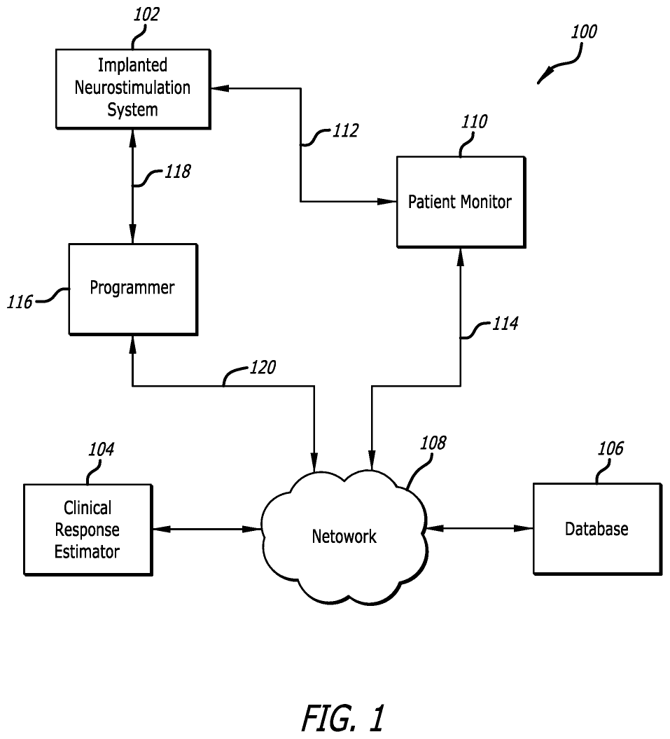

FIG. 1 is a block diagram illustration of a system 100 in which machined learned classification models, e.g., deep learning models, are applied to physiological information, e.g., EEG records, to classify electrographic seizures in terms of location and spread pattern. The system includes an implanted neurostimulation system 102, a records classification processor 104, and a database 106, each configured to provide and/or obtain a patient's physiological information over a network 108.

Physiological information corresponding to EEG records may be captured by the implanted neurostimulation system 102. As described later below, these EEG records may correspond to digitally recorded time series samples of electrocorticographic activity (e.g., a time series waveform). These EEG records may also be in another form or format derived from the time series samples. For example, an EEG record may be a spectrogram image or a time series waveform image of the brain electrical activity. (It will be appreciated that any time-series EEG can be represented as a spectrogram.) Alternatively, time-series waveforms may be directly used.

Other types of physiological information, e.g., pH levels, blood oxygen levels, neurotransmitters concentrations, heart rate, blood pressure, blood glucose levels, hormone levels, sleep states, posture, etc., may be captured and preserved by an implanted neurostimulation system 102 as physiological records. Collectively, the EEG records and other physiological records preserved by an implanted neurostimulation system 102 are part of a dataset for the patient in which the device is implanted. Non-physiological information, forms part of the dataset and may include records or files of patient demographics (e.g., age, gender), patient drug regimen (e.g., type of drug, dose, and time of day of dose), and patient clinical outcomes, such as the rate of electrographic seizure detection and electrographic seizure onset (e.g., as detected and recorded by the implanted neurostimulation system), the rate of clinical seizures (e.g., as reported in a seizure diary or detected based on accelerometer recordings).

The neurostimulation system 102 includes implantable components, namely, an active medical device or neurostimulator, and one or more electrode-bearing leads. The electrodes are configured to rest in or on neural tissue in the patient's brain when the leads are implanted. The neurostimulator may be configured to be implanted in or on the patient's cranium or elsewhere in the patient (e.g., pectorally). Once the neurostimulator is implanted, a proximal end of each lead is connected to the neurostimulator. The combination of the active implanted medical device and the implanted lead(s) is configurable to sense physiological signals from the brain and process and store records of the sensed signals. In this example, the physiological signals the electrodes sense and transmit through the lead(s) to the neurostimulator are electrocorticographic signals. The neurostimulator is configured to record samples or segments of the sensed EEGs, and to store them in a memory.

A neurostimulation system 102 may capture different data types based on EEG signals. Data types may be captured at different time scales. Some examples of data types captured by a neurostimulation system 102 include: (1) continuous recordings (EEG records) of raw brain data at a certain sampling rate such as 1000, 500 or 250 Hz, (2) continuous measures of derived brain data such as spectral power in certain frequency bands (example 1-4 Hz band, 4-8 Hz band, 8-2 Hz band, 12-25 Hz band, 25-50 Hz band, 50-90 Hz band and so on) computed in small moving and overlapping time windows such as 128, 256 or 512 milliseconds; (3) counts of abnormal events in bins of varying durations such as minutes, days or hours; (4) sampled raw time series or derived brain data that are saved at random time points, specific time points (preprogrammed by a physician for example) or are sampled in response to a trigger such as detection of abnormal events in brain or when a patient swipes a magnet over the neurostimulator; and (5) patient reports of outcomes. These are almost always not continuous and only intermittently available.

A neurostimulation system 102 may also be configured to sense and record other types of physiological signals besides EEG signals. To this end, the neurostimulation system 102 may include a lead as disclosed in U.S. Pat. No. 10,123,717, entitled Multimodal Brain Sensing Lead, which is herein incorporated by reference. Such a multimodal brain sensing lead may include: (1) macroelectrodes; (2) microelectrodes; (3) light emitters; and (4) photodetectors. Different sensing modalities of the implanted neurostimulation system 102 use the different transducers as follows: (1) neuronal field potential measurements are made using macroelectrodes; (2) neuronal single unit activity measurements are made using microelectrodes; (3) neuronal multi-unit activity measurements are also made using microelectrodes; (4) rheoencephalography measurements are made using macroelectrodes; (5) neurochemical and pharmaceutical voltammetric measurements are made using both macroelectrodes and microelectrodes; (6) optical blood flow and volume measurements are made using light emitters and photodetectors; and (7) optical blood oxygenation measurements are also made using light emitters and photodetectors.

Configured as such, the neurostimulation system 102 may sense and record signals indicative of blood oxygen level and blood volume in neural tissue, and signals indicative of chemical concentrations and neurotransmitter concentrations in neural tissue. From these signals, the neurostimulation system 102 may derive other physiological information. For example, blood flow, blood oxygenation, blood pressure, heart rate, and breathing rate may be estimated from blood oxygen and blood volume measurements, while pH levels and blood glucose levels may be derived from chemical concentrations and neurotransmitter concentrations.

The neurostimulation system 102 may also include one or more electrodes configured to sense electrical cardiac activity indicative of heart rate, a pressure sensor configured to provide signals indicative of blood pressure, an accelerometer configured to provide motion signals indicative of motion and the position of the patient. From these accelerometer signals, the implanted neurostimulation system 102 may derive other physiological information corresponding to clinical seizures, patient posture, and sleep state.

Other types of physiological information may be obtained and stored by the neurostimulation system 102 from sources independent of the neurostimulation system. For example, an external wearable device, e.g., patch, may include a sensor configured to sense and track cortisol levels, i.e., stress hormones, in sweat, while an external wearable device, e.g., watch, may include or a sensor configured to sense blood pressure. The physiological information from these external devices may be transmitted to the implanted neurostimulation system 102 for inclusion in the patient's dataset.

Records of physiological information may be generated by the neurostimulation system 102 based on an occurrence of an event or trigger. To this end, a neurostimulation system 102 can be configured to create an EEG record of a sensed EEG when an event the system is programmed to detect is detected. For example, the neurostimulation system 102 may be configured to detect an event corresponding to an electrographic seizure or the onset of an electrographic seizure from a sensed EEG, and to create an EEG record of the corresponding EEG signal spanning the time period 60 seconds before the event was detected and 30 seconds thereafter. The neurostimulation system 102 can also be programmed to create an EEG record of a sensed EEG at certain times of day (e.g., at noon and at midnight). These are sometimes referred to as “scheduled EEGs.” In addition, the neurostimulation system 102 may be configured to store an EEG record upon some other trigger, such as when the patient swipes a magnet over the location on the patient's body at which the neurostimulator is implanted (the patient might be instructed to do this whenever he or she thinks a seizure is coming on).

The neurostimulation system 102 can also be programmed to designate EEG records based on the event that triggered its recording and to include that designation in the EEG record. For example, EEG records resulting from the detection of abnormal electrical activity, e.g., an electrographic seizure or the onset of an electrographic seizure, may be marked as such, while EEG records EEGs that do not reflect abnormal activity may be designated as baseline EEG records. Thus, for a given patient, a dataset may contain EEG records corresponding to what is happening in the patient's brain during and around when an event occurs, scheduled EEG records acquired at a particular time, and EEG records stored by the neurostimulator when a patient triggers storage with a magnet. Some of these EEG records, especially the ones recorded at the time of an event or when triggered by a magnet swipe, may reflect the patient's electrographic seizures. The dataset may include information or a data type about whatever triggered the neurostimulator to store a given EEG, such as the type of event (e.g., Pattern “A” or Pattern “B,” a magnet swipe) or the time of day (e.g., scheduled EEG).

Typically, some sort of linkage or mapping among the various types of physiological information is provided in a dataset. To this end, each record may have one or more associated tags or parameters. For example, physiological records may have a time stamp that allows a set of physiological records at a given point in time to be located for processing. Physiological records may have a tag that indicates the basis, e.g., seizure detection, magnet swipe, scheduled time of day, for preserving the record. These tags allow a set of physiological records to be selected for processing based on a single criterion or a combination of criteria. Other tags may include day of capture, area of the brain at which the electrical activity was captured, basis for record creation (e.g., seizure detection, scheduled, patient initiated), characteristic of the record (e.g., power spectral density of EEG signal prior to stimulation).

Once created by a neurostimulation system 102, physiological records stored in the system can be relayed elsewhere, such as to an external component like the database 106 either directly or through an interim external component. For example, the patient monitor 110 can be used with an accessory (not shown) to establish a communications link 112 with the implanted neurostimulator (e.g., a short-range telemetry link), which allows records stored on the neurostimulator to be transmitted to the patient monitor 110. Once on the patient monitor, the physiological records can be transmitted to the database 106 via the network 108 over a communication link 114 (which may comprise a physical, WiFi, or cellular internet transmission).

Alternatively, the clinician may be provided with an external component, such as a programmer 116 that, like the patient monitor 110, is configured to establish a communications link 118 with the implanted neurostimulator. The clinician can use the programmer to adjust the programmable parameters of the neurostimulator (e.g., the parameters that govern the electrical stimulation waveform that is used for therapy). The programmer 116 is able to specify and set variable parameters in the implanted neurostimulator 202 (e.g., detection parameter sets and stimulation parameter sets) to adapt the function of the device to meet the patient's needs, upload or receive data from the neurostimulation system to the programmer, download or transmit program code and other information from the programmer to the neurostimulator, or command the neurostimulator to perform specific actions or change modes as desired by a physician operating the programmer.

The programmer 116 also may be used to display the real time EEG signals being sensed by the electrodes from the patient and to store them on the programmer. It also can be used like the patient monitor 110 to acquire physiological records that have been stored by the neurostimulator since the last time the neurostimulator was “interrogated” for those records by either a patient monitor 110 or programmer. As is the case with a patient monitor 110, once physiological records are stored on a programmer 116, they can be transmitted via a communication link 120 and the network 108 to other components of the system 100, such as the database 106 and/or the records classification processor 104 (either directly or via the database 106).

A neurostimulation system 102 may be configured to deliver electrical stimulation therapy in response to “events” that the neurostimulator is configured to detect. An event may be defined for the neurostimulator by setting the values of programmable detection parameters such that when a pattern corresponding to a pattern defined by the detection parameters occurs in the monitored EEG signals, the occurrence of that pattern will be detected as an event. Other implantable neurostimulation systems that might be used in the subject system may not have this feature of responsive neurostimulation at all or may not have it enabled.

While FIG. 1 illustrates a single implanted neurostimulation system 102 and patient monitor 110 and programmer 116, numerous neurostimulation systems implanted across a patient population may access the network 108 to provide patient physiological records and patient information to the records classification processor 104 and the database 106. Accordingly, the system 100 can provide access to tens of thousands of patient EEG records.

Overview of Implanted Neurostimulation System

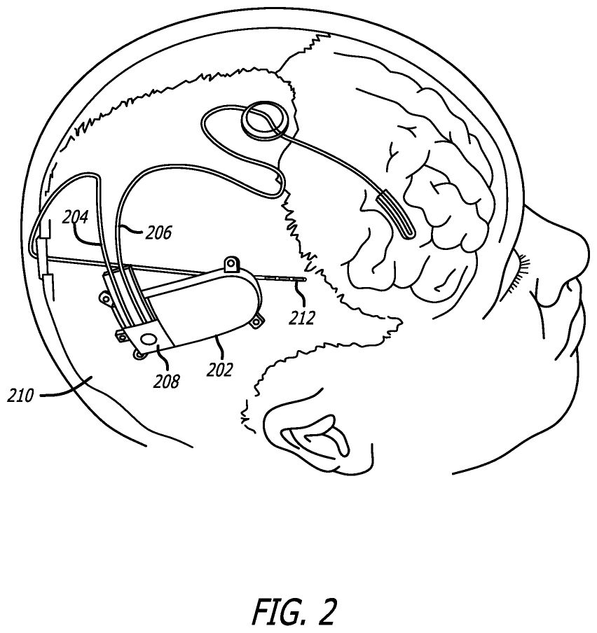

FIG. 2 is an illustration of the implanted neurostimulation system including a neurostimulator 202 and two electrode-bearing brain leads 204, 206, implanted in a patient. The system is configured to sense and record electrical brain activity and other physiological information and provide such records as part of the system of FIG. 1.

The neurostimulator 202 includes a lead connector 208 adapted to receive one or more of the brain leads, such as a deep brain or depth lead 204 and a cortical strip lead 206. The depth lead is implanted so that a distal end of it is situated within the patient's neural tissue, whereas the cortical strip lead is implanted under the dura mater so that a distal end of it rests on a surface of the brain. The lead connector 208 acts to physically secure the brain leads 204, 206 to the neurostimulator 202, and facilitates electrical connection to conductors in the brain leads 204, 206 coupling one or more electrodes at or near a distal end of the lead to circuitry within the neurostimulator 202.

The proximal portion of the deep brain lead 204 is generally situated on the outer surface of the cranium 210 (and under the patient's scalp), while the distal portion of the lead enters the cranium and is coupled to at least one depth electrode 212 implanted in a desired location in the patient's brain. The proximal portion of the cortical lead 206 is generally situated on the outer surface of the cranium 210 (and under the patient's scalp), while the distal portion of the lead enters the cranium. The distal portion of the cortical lead 206 includes at least one cortical electrode (not visible) implanted in a desired location on the patient's brain.

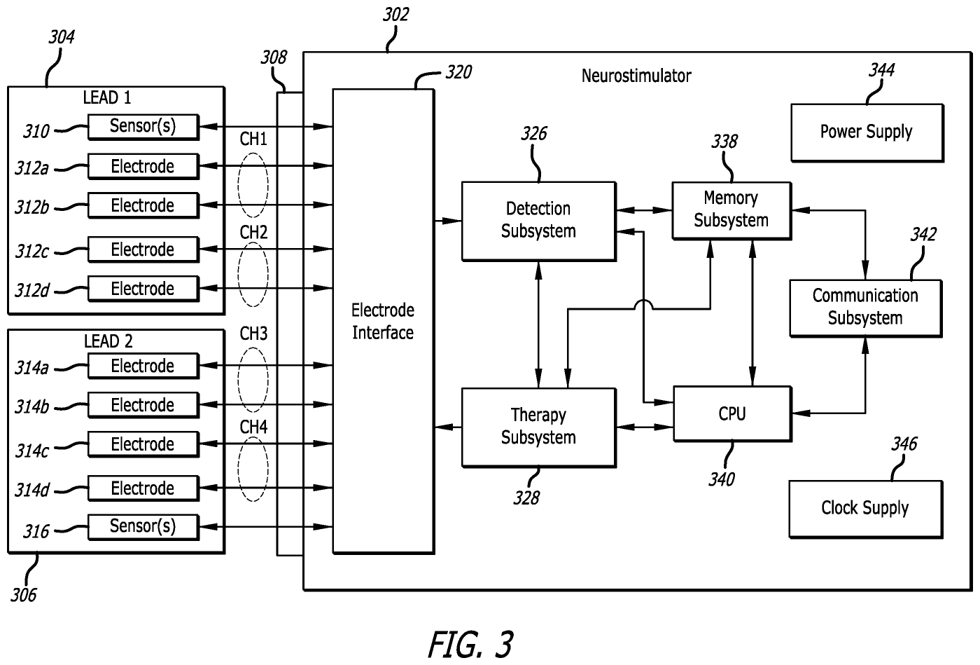

FIG. 3 is a block diagram of the implanted neurostimulation system of FIG. 2. The system may be configured to sense electrical brain activity, detect neurological events in accordance with a set of detection parameters, deliver electrical neurostimulation to the brain in accordance with a set of stimulation parameters, and store records of electrical brain activity and other physiological information for transmission to the database 106 of the system of FIG. 1.

The neurostimulator 302 includes a lead connector 308 adapted to receive a connector end of each brain lead 304, 306, to thereby electrically couple each lead and its associated electrodes 312a-d, 314a-d with the neurostimulator. The neurostimulator 302 may configure an electrode 312a-d, 314a-d as either a sensor (for purposes of sensing electrical activity of the brain) or a stimulator (for purposes of delivering therapy to the patient in the form of electrical stimulation) or both.

The electrodes 312a-d, 314a-d are connected to an electrode interface 320. The electrode interface 320 can select each electrode 312a-d, 314a-d as required for sensing and stimulation. The electrode interface 320 may also provide other features, capabilities, or aspects, including but not limited to amplification, isolation, and charge-balancing functions, that are required for a proper interface with neurological tissue. The electrode interface 320 is coupled to a detection subsystem 326, which is configured to process electrical activity of the brain sensed through the electrode 312a-d, 314a-d. The electrode interface 320 may also be coupled to a therapy subsystem 328, which is configured to deliver therapy to the patient through the electrode 312a-d, 314a-d in the form of electrical stimulation.

One or both of the brain leads 304, 306 may have one or more physiological sensors 310, 316 that enable the capture and recording of other types of physiological information, e.g., pH levels, blood oxygen levels, neurotransmitters concentrations, heart rate, blood pressure, blood glucose levels, hormone levels, sleep states, posture, etc. To this end, one or both of the brain leads 304, 306 may be configured as disclosed in U.S. Pat. No. 10,123,717, entitled Multimodal Brain Sensing Lead, which is herein incorporated by reference, and the one or more physiological sensors 310, 316 may correspond to different transducers, e.g., macroelectrodes, microelectrodes, light emitters, and photodetectors that enable different sensing modalities.

The neurostimulator 302 includes a memory subsystem 338 and a central processing unit (CPU) 340, which can take the form of a microcontroller. The memory subsystem 338 is coupled to the detection subsystem 326, and may receive and store records of data representative of sensed electrographic signals for transmission to the system of FIG. 1. The memory subsystem 338 is also coupled to the therapy subsystem 328 and the CPU 340. In addition to the memory subsystem 338, the CPU 340 is also connected to the detection subsystem 326 and the therapy subsystem 328 for direct control of those subsystems.

The neurostimulator 302 also includes a communication subsystem 342. The communication subsystem 342 enables communication between the neurostimulator 302 and an external device, such as a programmer 116 or patient monitor 110, through a wireless communication link. As described above with reference to FIG. 1, the programmer 116 allows a clinician to read out records of patient data, as well as ancillary information associated with those records. The neurostimulator 302 also includes a power supply 344 and a clock supply 346. The power supply 344 supplies the voltages and currents necessary for each of the other subsystems. The clock supply 346 supplies substantially all the other subsystems with any clock and timing signals necessary for their operation.

Physiological Information

With respect to physiological information, a dataset may include records or files of physiological information corresponding to electrical activity of the brain. Hereinafter, electrical activity of the brain is referred to as an “EEG,” the digital representation, i.e., stored data bits, of electrical activity of the brain is referred to as “EEG data,” and a physiological record corresponding to electrical activity of a patient's brain is referred to as an “EEG record.” It will be understood that EEG includes electrical activity sensed directly from the neural tissue, which sometimes is referred to as electrocorticographic activity, an electrocorticogram, or “ECoG.”

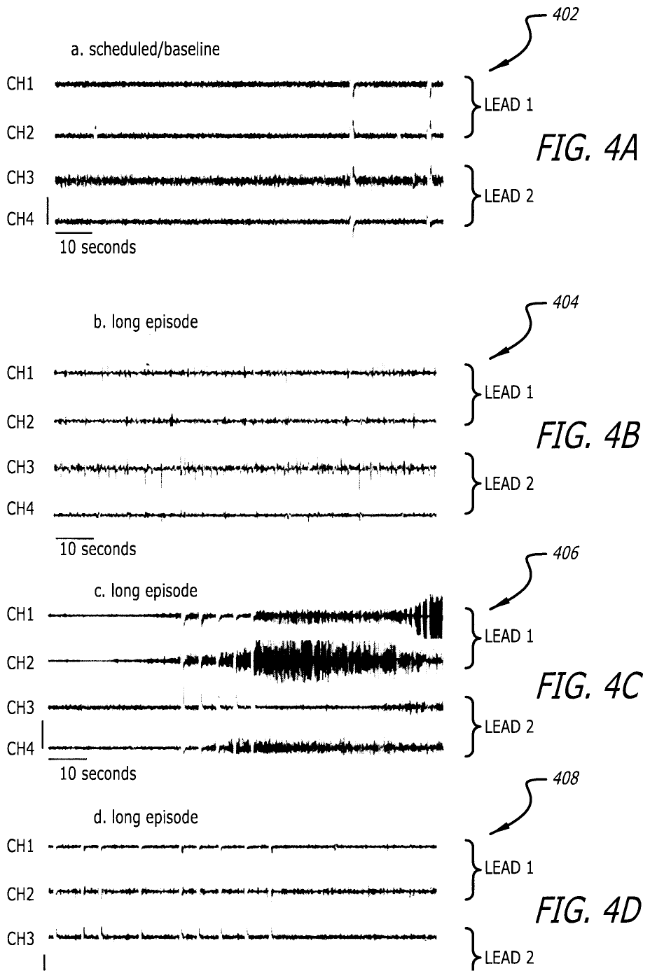

An EEG record corresponding to electrical activity of a patient's brain may be visualized in the form of a time series waveform image. For example, with reference to FIGS. 4A-4D, an EEG record 402, 404, 406, 408 may consist of four channels (CH1, CH2, CH3, CH4) of EEG data, each visualized as a separate time series waveform, where two channels (CH1 and CH2) are associated with a first lead (LEAD1), and two channels (CH3 and CH4) are associated with a second lead (LEAD2). These four separate time series waveforms (and their corresponding EEG data) collectively represent the EEG record 402, 404, 406, 408. In some embodiments, the systems and methods disclosed herein process EEG records 402, 404, 406, 408 on a per channel basis. Accordingly, each channel time series waveform (and it corresponding EEG data) may also be referred to herein as an EEG record.

The EEC records illustrated in FIGS. 4A-4D are examples of types of EEG data captured by the system in one example patient. As described above with reference to FIG. 3, the neurostimulator that can be connected to up to two leads. The leads may be either strip or depth leads. EEG files captured by the neurostimulator typically contain four channels of data. Channels 1 and 2 typically belong to lead 1, and channels 3 and 4 typically belong to lead 2.

The two main types of EEG records captured and stored by the device include scheduled EEG records (FIG. 4A), and long episode EEG records (FIGS. 4B-4D). Scheduled EEG records generally contain background non-seizure activity, and its storage is triggered by time of day. Long episode EEG records contain long trains of abnormal EEG activity which can sometimes be electrographic seizures. Automatic capture and storage of long episodes EEG records is triggered when long trains of abnormal events are sensed by the neurostimulator.

While the methods and systems disclosed herein are primarily described with reference to EEG records in the form of time series waveform images, other forms of EEG records may be used. For example, EEG records in the form of spectrograms may be processed by the methods and systems. Furthermore, while the methods and systems disclosed herein are primarily described with reference to records comprising electrical activity of the brain, it will be appreciated that other physiological information and non-physiological information may be processed.

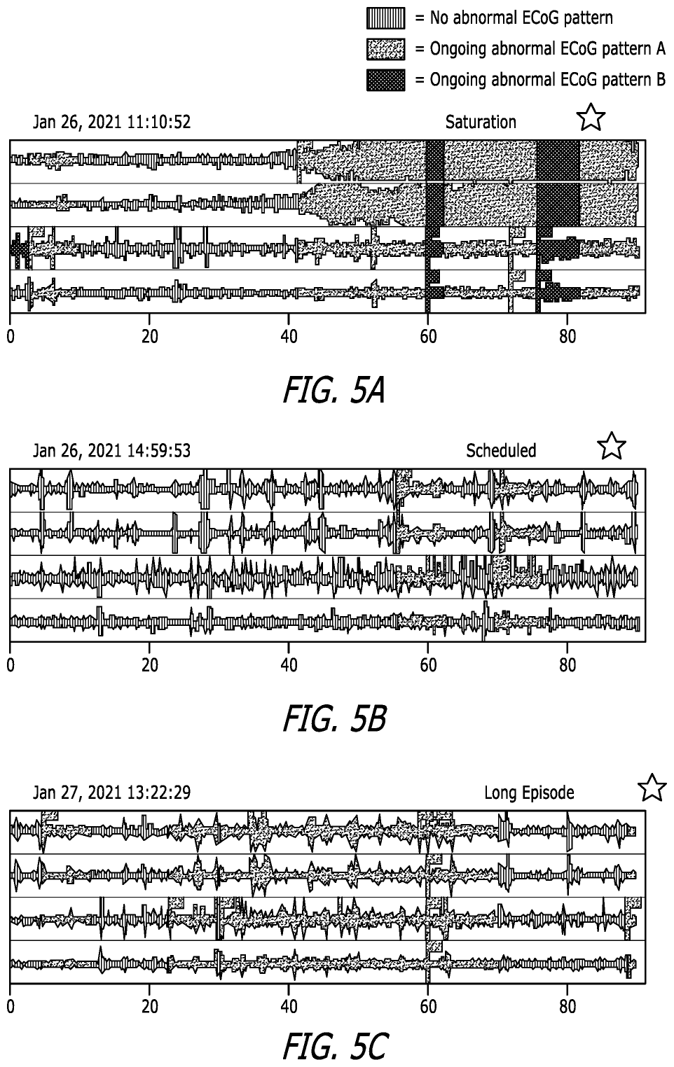

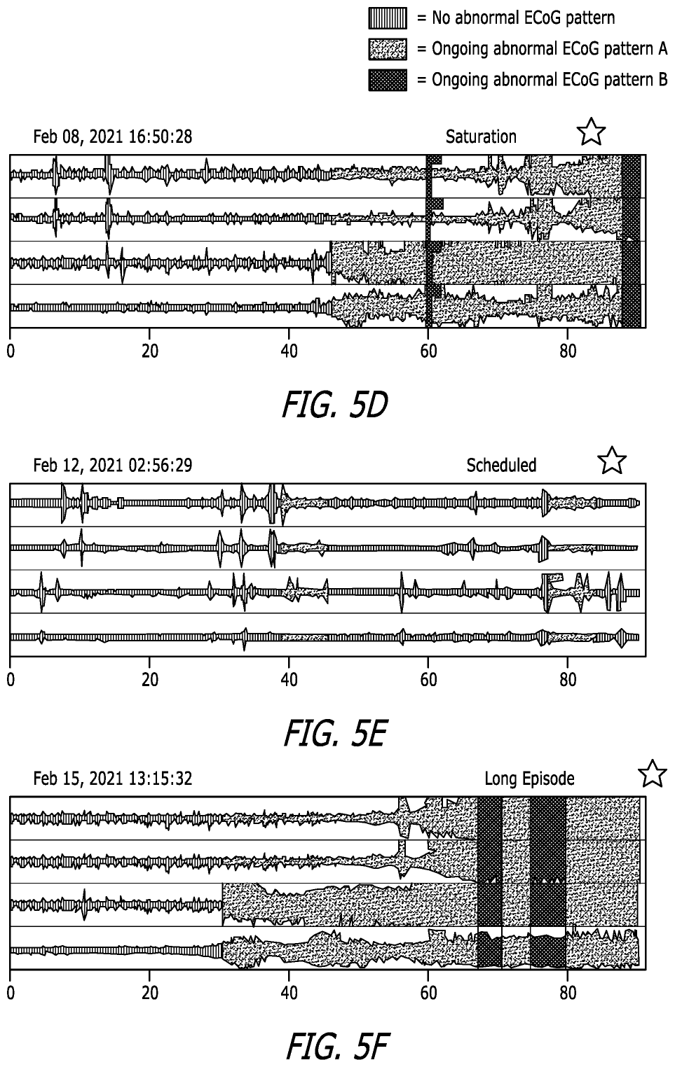

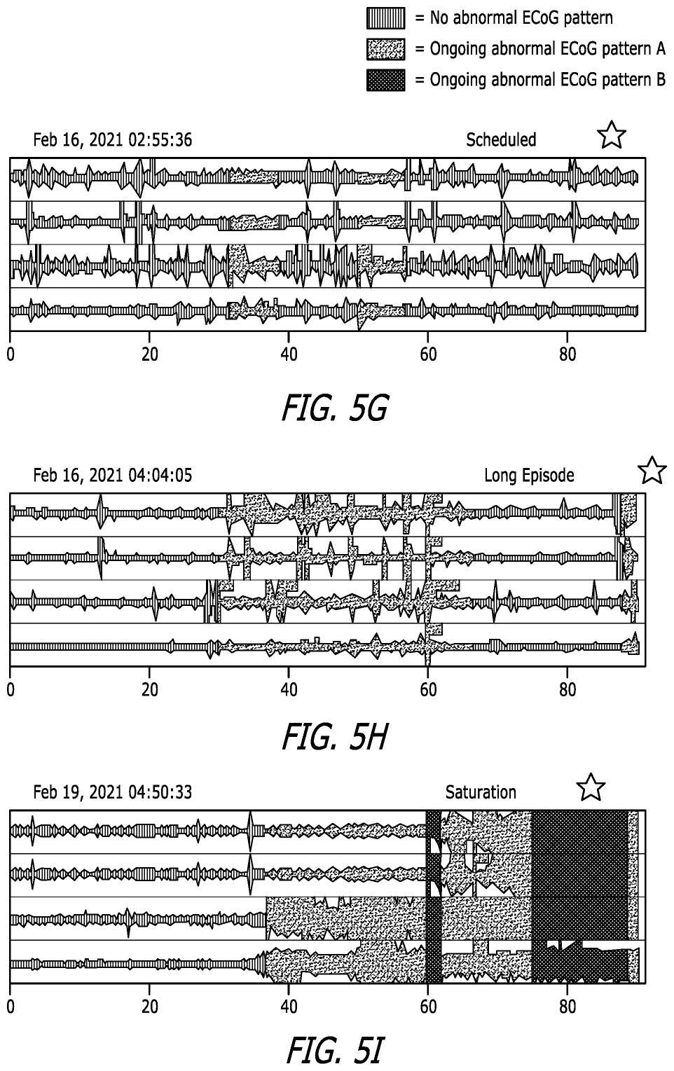

With reference to FIGS. 5A-5I, in a conventional review of a patient's records, EEG records are visually presented in chronological order to a physician for manual review. The physician may be able to sort by the EEG records by trigger reason such as scheduled EEG records, or long episode EEG records. Typically, hundreds and thousands of EEG records of each trigger reason are captured in patients. Physicians may visually scroll through these EEG records to identify ones of interest (typically those containing electrographic seizures). Additionally, through visual observation physicians may manually sort EEG records into distinct categories based on the seizure onset channel or lead, the activity pattern at the seizure onsets, and the time of spread of the seizures in the EEG records. Counts of EEG records belonging to the different classes/categories during different programming epochs may be used by the physician to determine the efficacy of brain electrical stimulation and antiepileptic drugs, and also to determine next treatment steps.

Records Classification Processor

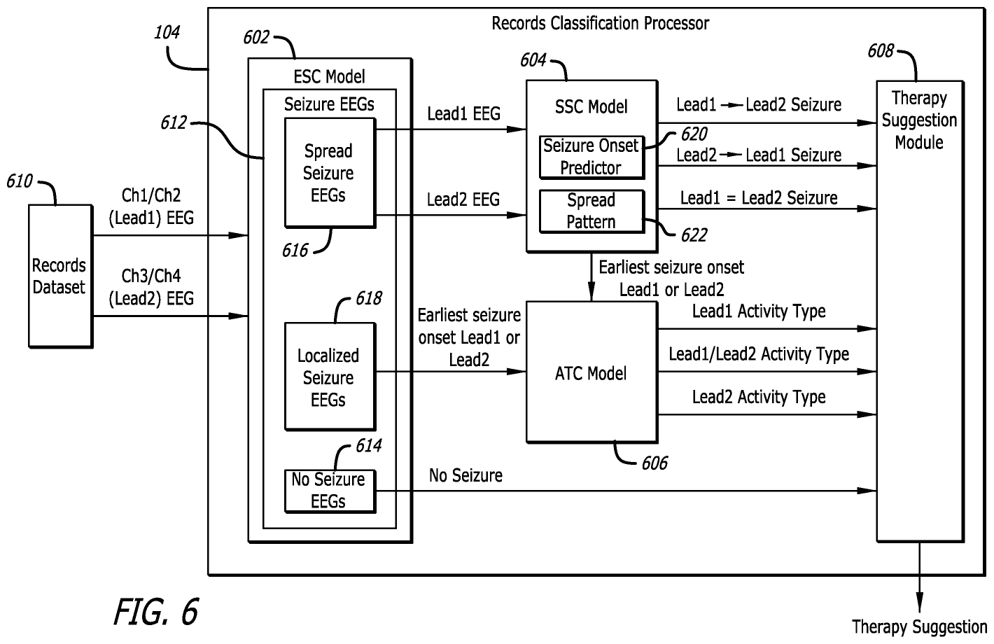

With reference to FIG. 6, in accordance with the present disclosure, the records classification processor 104 is configured to: 1) automatically classify EEG records into sets of seizures EEG records and non-seizure EEG records, and to further classify seizure EEG records into one or more distinct seizure categories, such as local seizures and spread seizures, and 2) to automatically classify the seizure EEG records within one or more of the distinct seizure categories, in terms of seizure spread patterns (based on predicted times of seizure onset) and/or in terms of seizure onset type (or activity type).

The records classification processor 104 includes an electrographic seizure classifier 602 (also referred to as an ESC model), a seizure onset classifier 604 (also referred to as an SSC model), an activity type classifier 606 (also referred to as an ATC model), and a treatment module 608. The records classification processor 104 is configured to interface with a records dataset 610, which may be part of the database 106 of FIG. 1, for purposes of receiving EEG records for processing. The records classification processor 104 also interfaces with a display (not shown) to enable the display of EEG records and the display of EEG record classifications, e.g., seizure category, seizure spread pattern, and seizure onset type, as determined by the records classification processor. The records classification processor 104 may also interface with a user interface (not shown) to receive inputs from expert users.

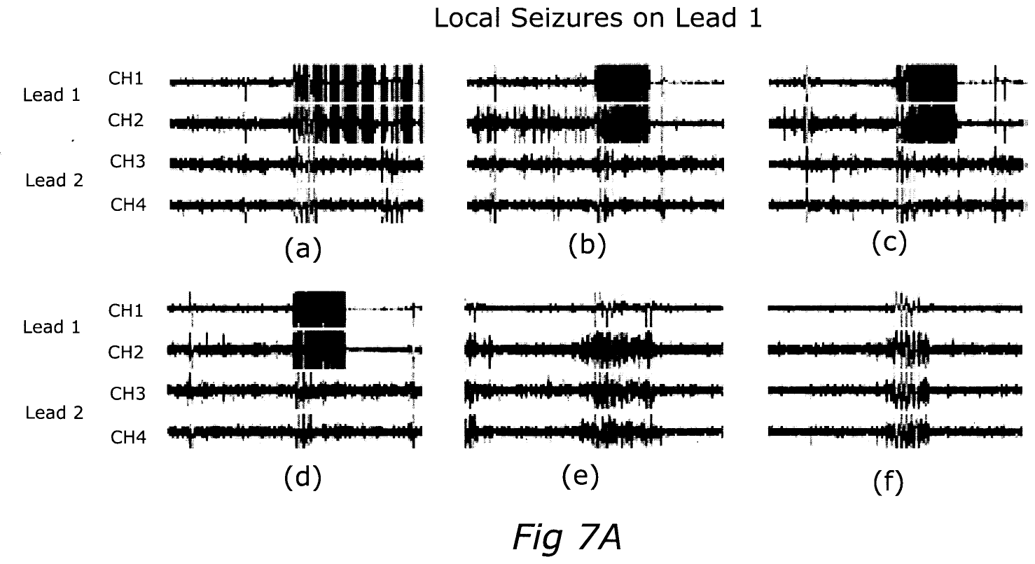

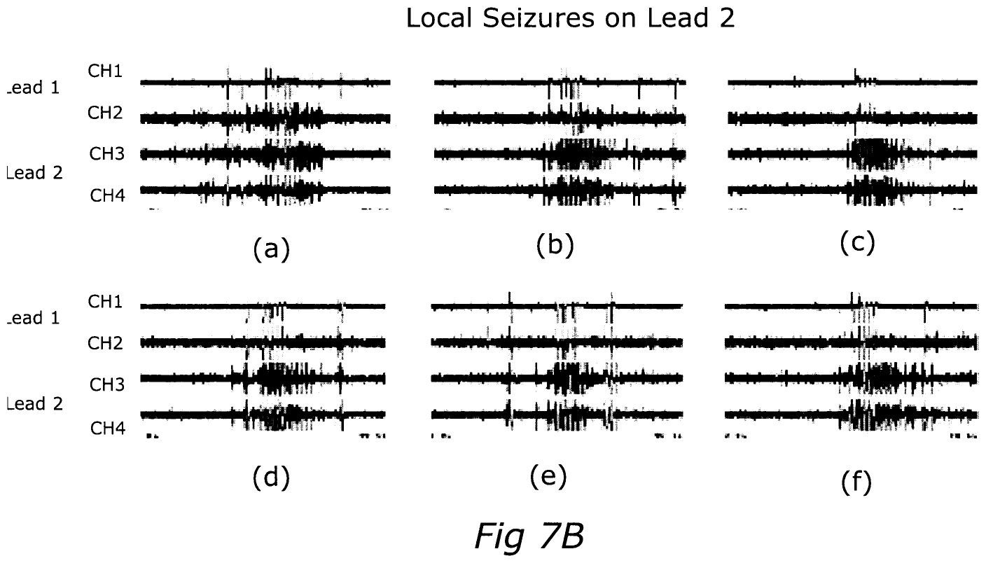

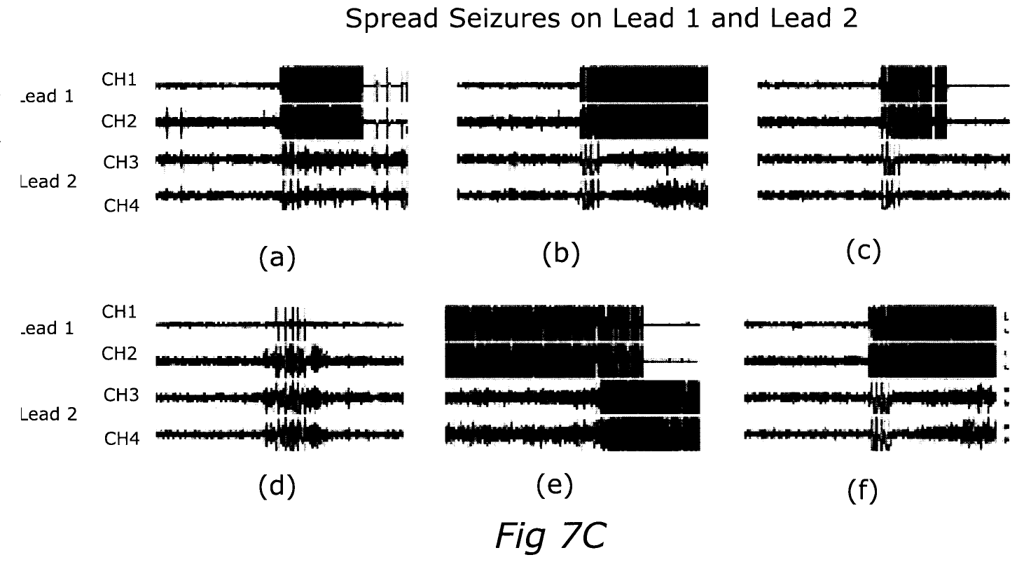

With reference to FIGS. 6, 7A, 7B, and 7C, one approach to increasing the efficiency of the EEG record review process is to automate the sorting/classifying/categorizing of a patient's EEG records. The ESC model 602, SSC model 604, and ATC model 606 or combinations thereof, are configured to determine a number of classes or categories for each patient based on that patient's EEG records. The number of categories may vary from patient to patient. For example, in a patient with fairly stereotypical brain electrical activity patterns the ESC model 602 may classify an EEG record in one of two categories or classes, such as seizure and non-seizure categories. In the case of a patient with many different types of brain activity patterns, the combination of the ESC model 602, the SSC model 604, and the ATC model 606 may classify an EEG record in more than two categories, such as low voltage fast onset electrographic seizures starting on the left lead, hypersynchronous spiking onset electrographic seizures starting on the right lead, irregular beta activity onset electrographic seizures starting on the left lead, and so on.

The determination of the number of categories for each patient is based on activity in the EEG records captured in that patient. For example, with reference to the EEG records shown in FIGS. 7A, 7B, and 7C, the ESC model 602 and the SSC model 604 may be trained to determine that seizure EEG records for a patient fall into one of the following three distinct seizure categories:

1) A local seizure on lead 1. This corresponds to a seizure onset detection in the Ch1/Ch2 (Lead 1) EEG, without a seizure onset detection in the Ch3/Ch4 (Lead 2) EEG). In other words, a seizure is on lead 1 only. Examples of these types of EEG records are shown in FIG. 7A.

2) A local seizure on lead 2. This corresponds to a seizure onset detection in the Ch3/Ch4 (Lead 2) EEG, without a seizure onset detection in the Ch1/Ch2 (Lead 1) EEG. In other words, a seizure is on lead 2 only. Examples of these types of EEG records are shown in FIG. 7B.

3) A spread seizure on lead 1 and lead 2. This corresponds to a seizure onset detection in the Ch1/Ch2 (Lead 1) EEG, and a seizure onset detection in the Ch3/Ch4 (Lead 2) EEG. Examples of these types of records are shown in FIG. 7C.

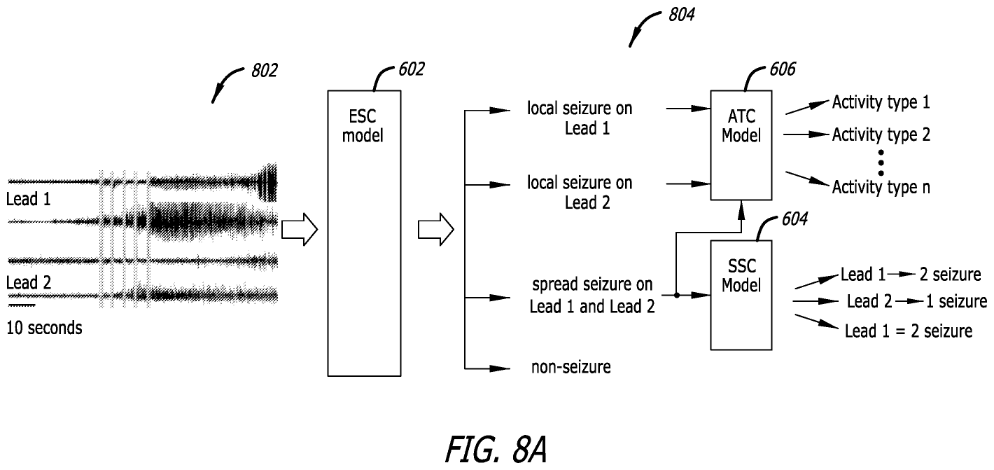

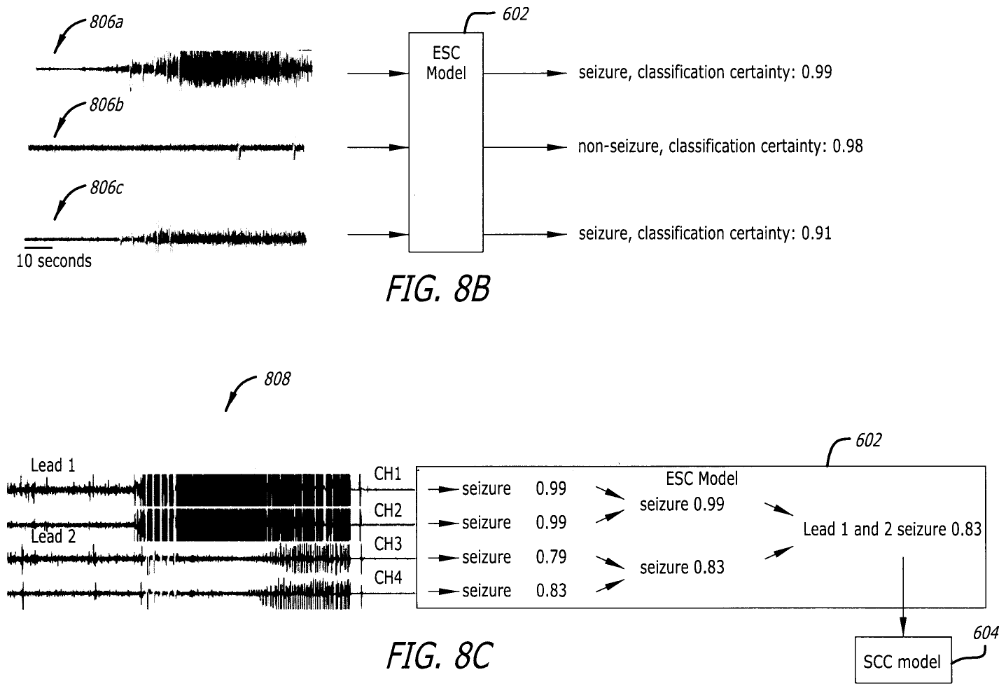

With reference to FIGS. 6, 8A, 8B and 8C, the sequence of machine learning/deep learning models of the records classification processor 104 include the ESC model 602 and the SSC model 604. In operation of the records classification processor 104, the ESC model 602 is applied to an EEG record (in this case the EEG record includes all four channels). With reference to FIG. 8A, the ESC model 602 classifies an EEG record 802 into one of four categories 804 based on the leads containing the electrographic seizure. The four categories include the three above described categories: (1) local seizure on lead 1, (2) local seizure on lead 2, (3) spread seizure on lead 1 and lead 2, and an additional category: (4) non-seizure. Note that in FIG. 8A, while all possible categories 804 are listed, the actual EEG record 802 shown in the figure is determined to correspond to a spread seizure lead 1 and lead 2.

The ESC model 602 may correspond to a machine-learned model trained in accordance with one of the models or a combination of models described in Barry W, Arcot Desai S, Tcheng TK and Morrell MJ (2021), A High Accuracy Electrographic Seizure Classifier Trained Using Semi-Supervised Labeling Applied to a Large Spectrogram Dataset. Front. Neurosci. June 2021, Vol. 15, Art. 667373, which is incorporated by reference. For example, a large dataset of EEG records from over hundred patients can be manually labeled as seizures and non-seizures and used to train a convolutional neural network (CNN) based model. One such trained model described in the Barry et al. had a test classification accuracy of >95% on individual EEG channels which means the trained model could classify EEG channels from new patients as seizures and non-seizures with over 95% classification accuracy.

With reference to FIG. 6, the ESC model 602 may be configured to perform a first level of classification in which EEG records are classified as either a seizure EEG record 612 or non-seizure EEG 614 record. The ESC model 602 also may be configured to perform a second level of classification on seizure EEG records 612 to further classify each seizure EEG record as one of a spread-seizure record EEG 616 or a local-seizure EEG record 618.

Regarding the first level of classification and with reference to FIG. 8B, the ESC model 602 may be individually applied to channel EEG records 806a, 806, 806c to provide a seizure classification and a measure of certainty of such classification. In the example of FIG. 8B, channel EEG record 806a is classified as a seizure, with a certainty of 0.99, channel EEG record 806b is classified as a non-seizure with a certainty of 0.98, and channel EEG record 806c is classified as a seizure with a certainty of 0.91.

With reference to FIG. 8C, individual channel EEG record classifications may be combined to derive an overall classification for an EEG record 808. In the example of FIG. 8C, the EEG record 808 as a whole is classified as a spread seizure on lead 1 and lead 2, with a certainty of 0.83. To this end and starting from the left of FIG. 8C, EEG records sensed at CH1 of Lead 1 and CH2 of Lead 1 are individually classified as seizure or non-seizure by the ESC model 602. EEG records sensed at CH3 of Lead 2 and CH4 of Lead 2 are also individually classified as a seizure or non-seizure. In the example of FIG. 8C, all channels are classified as seizures.

Next, the combination of the CH1 and CH2 EEG records sensed by Lead 1 are classified by the ESC model 602 based on the following criteria: if either CH1 or CH2 or both CH1 and CH2 are classified as a seizure, then the Lead 1 EEG record is classified as a seizure; otherwise, the Lead 1 EEG record is classified as a non-seizure. In the example EEGs in FIG. 8C, both CH1 and CH2 are classified as a seizure. Thus, the Lead 1 EEG record is classified as a seizure EEG record. Similarly, the combination of the CH3 and CH4 EEG records sensed and recorded by Lead 2 are classified based on the following criteria: if either CH3 or CH4 or both CH3 and CH4 are classified as a seizure, then the Lead 2 EEG records is classified as a seizure; otherwise, the Lead 2 EEG record is classified as a non-seizure. In the example EEGs in FIG. 8C, both CH3 and CH4 are classified as a seizure. Thus, the Lead 2 EEG record is classified as a seizure EEG record.

The foregoing classification of each of the Lead 1 EEG record and the Lead 2 EEG record as either a seizure record or non-seizure record serves as a basis for a second level of classification that further classifies a seizure EEG record 612 as one of a spread-seizure EEG record 616 or a local-seizure EEG record 618. More specifically, a case where both of the Lead 1 EEG record and the Lead 2 EEG record are classified as seizures is further classified by the ESC model 602 as spread-seizure EEG record 616, e.g., a spread seizure on lead 1 and lead 2. This is the example shown in FIG. 8C. However, a case where only one of Lead 1 EEG record or the Lead 2 EEG record is classified as a seizure is further classified by the ESC model 602 as a local-seizure EEG record 618, e.g., a local seizure on lead 1 or a local seizure on lead 2.

Returning to FIGS. 6 and 8A, the SSC model 604 is applied to EEG records classified as spread seizures on lead 1 and lead 2. EEG records classified as such are referred to as spread-seizure EEG records. The SSC model 604 is configured to identify seizure spread patterns in spread-seizure EEG records and to classify these EEG records based on their seizure spread.

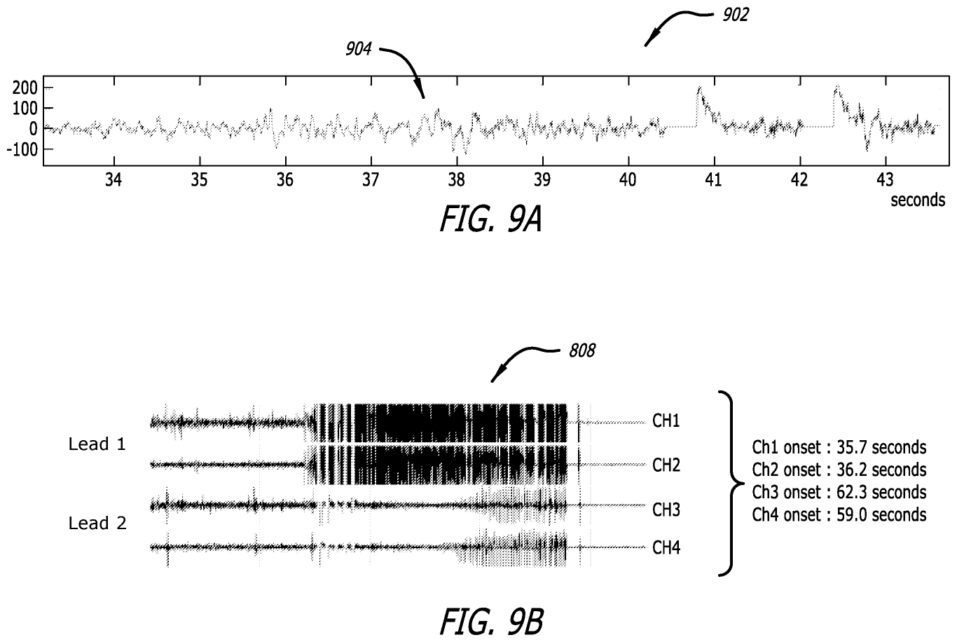

With reference to FIGS. 6, 9A, and 9B, the SSC model 604 includes a seizure onset predictor 620. The seizure onset predictor 620 may be a deep learning model configured to predict a seizure onset 904 on individual channel EEG records 902 and to output a corresponding seizure onset time. For example, in FIG. 9B, a Lead 1 CH1 EEG record is determined to have a seizure onset time of 35.7 seconds, a Lead 1 CH2 EEG record is determined to have a seizure onset time of 36.2 seconds, a Lead 2 CH3 EEG record is determined to have a seizure onset time of 62.3 seconds, and a Lead 2 CH4 EEG record is determined to have a seizure onset time of 59.0 seconds.

In some embodiments, the seizure onset predictor 620 of the SSC model 604 comprises a convolutional neural network (CNN) based architecture. In this architecture, a large dataset of EEG records is manually labeled for seizure onset times on individual EEG channels and a CNN is trained on the labeled dataset. The trained CNN is applied to spread-seizure EEG records to predict seizure onsets and provide times of seizure onsets. In other embodiments, the seizure onset predictor 620 deep learning model may be created using different methods, such as attention models (or other sequence based models) or signal processing algorithms. See, for example, “Recurrent Neural Networks for Forecasting Epileptiform Electrographic Activity 24 Hours in Advance,” AES abstract no. 2.050, published Nov. 6, 2018.

With reference to FIGS. 6 and 8A, the SSC model 604 also includes a spread pattern module 622 that is configured to determine a spread pattern classification based on the predicted times of seizure onsets in the EEG record determined by the seizure onset predictor 620 deep learning model. The spread pattern classification determined by the spread pattern module 622 of the SSC model 604 may be one of the following:

1) A lead1-to-Lead2 seizure spread pattern (corresponding to an instance where the predicted time of seizure onset in the Lead1 EEG record precedes the predicted of time seizure onset in the Lead2 EEG record). An example of this type of EEG record is shown in FIG. 7C, panel (e).

2) A Lead2-to-lead 1 seizure spread pattern (corresponding to an instance where the predicted time of seizure onset in the Lead2 EEG record precedes the predicted time of seizure onset in the Lead1 EEG record).

3) A lead1-equals-Lead2 seizure spread pattern or “no spread pattern” (corresponding to an instance where the predicted time of seizure onset in the Lead1 EEG record is within a specified range of the predicted seizure onset in the Lead2 EEG record, for example if the onsets are within 2 seconds of each other). An example of this type of EEG record is shown in FIG. 7C, panels (b) and (f).

In instances of a lead1-to-Lead2 seizure spread pattern or a Lead2-to-Lead1 seizure spread pattern, the relevant EEG record may also be classified into a time category based on the time of spread of the seizure from one lead to the other. In one example configuration, the time categories include:

1) A fast spread. This corresponds to a time that is less than 10 seconds spread,

2) A medium spread. This corresponds to a time that is between 10 and 20 seconds, and

3) A slow spread. This corresponds to a time that is greater than 20 seconds spread.

To this end, the spread pattern module 622 of the seizure onset classifier 604 is configured to use the seizure onset times predicted by the seizure onset predictor 620 to calculate the time between the earliest seizure onset for the EEG record and the latest seizure onset for the EEG record, and to categorize the record accordingly. For example, with reference to FIG. 9B, the earliest time of seizure onset (37.7 seconds) in the EEG record 808 is on Lead 1 CH1, while the latest time of seizure onset (62.3 seconds) is on Lead2 CH3. Based on these times, the time of spread from this EEG record 808 is 26.6 seconds, which falls into the above-defined slow spread category.

With reference to FIGS. 6 and 8A, EEG records classified as a local seizure on lead 1 (also referred to as a Lead 1 seizure only), a local seizure on lead 2 (also referred to as a Lead 2 seizure only), or spread seizure on lead 1 and lead 2 (also referred to as a Lead 1 and 2 seizure) can be further classified based on seizure onset type or pattern by the ATC model 606. To this end, the ATC model 606 is configured to determine a seizure onset type based on electrical activity at the seizure onset in local-seizure EEG records and spread-seizure EEG records.

Accordingly, in some embodiments, for a given seizure record (local-seizure or spread seizure) the ATC model 606 is applied to the EEG record having the channel/lead with the earliest seizure onset. For example, considering local seizure records 618 identified by the ESC model 602 in FIG. 6, and with reference to the lead1-to-lead2 local-seizure records in FIG. 7A and the lead2-to-lead1 local seizures in FIG. 7B, the ATC model 606 may be applied to only the CH1/CH2 (Lead 1) EEG records of the records in FIG. 7A, and to only the CH3/CH4 (Lead 2) EEG records of the records in FIG. 7B.

Considering spread-seizure records 616 identified by the ESC model 602 and processed by the SSC model 604 in FIG. 6, and with reference to FIG. 7C, the ATC model 606 may be applied to the CH1/CH2 (Lead 1) EEG records when the spread pattern determined by the SSC model 604 is a lead1-to-lead2 seizure spread pattern (meaning the earliest seizure onset is on lead 1), and to the CH3/CH4 (Lead 2) EEG records when the spread pattern determined by the SSC model 604 is a lead2-to-lead1 seizure spread pattern (meaning the earliest seizure onset is on lead 2).

With regard to local-seizure EEG records and spread-seizure EEG records, the ATC model 606 may invoke a clustering method in which features are extracted from the EEG records and passed through a clustering algorithm such as Bayesian Gaussian Mixture Models as disclosed in Barry et al. to determine which type/pattern (or cluster) of seizure onset the local-seizure EEG records falls within. The ATC model 606 may be trained to recognize various type/patterns of electrical activity in EEG records corresponding to seizure onsets. Some examples are shown in the table below along with a description. EEG examples with these activity patterns are further described in Nune et al., “Treatment of drug-resistant epilepsy in patients with periventricular nodular heterotopia using RNS System: Efficacy and description of chronic electrophysiological recordings,” Clinical Neurophysiology 2019, which is incorporated by reference.

| Seizure Onset Type | Description |
|---|---|
| Low Voltage Fast | Slowly evolving, high frequency (>13 Hz) and low |
| (LVF) | amplitude activity progressing to lower frequencies |
| | and higher amplitude |
| Hypersynchronous | Periodic spiking at less than 3 Hz for a duration |
| (Hypersync) | of >10 seconds prior to seizure onset |
| Attenuation (Atten) | Voltage suppression without a significant increase in |
| | power at any frequency |
| Multiple (M) | Multiple concurrent onset types |
| Rhythmic Delta (D) | Rhythmic activity at 1 to <4 Hz or periodic spiking |
| | at >3 and <4 Hz |
| Rhythmic Theta (T) | Rhythmic 4 to <8 Hz activity |
| Rhythmic Alpha (A) | Rhythmic 8 Hz to <13 Hz activity |
| Semi-Rhythmic | High but variable amplitude, semi-rhythmic spiking |
| Beta (SRB) | in the beta frequency range |

As an alternative to a clustering algorithm, the ATC model 606 may invoke a rule based method in which features extracted from the EEG records are used to classify a seizure based on pre-set rules. Such rule based methods are disclosed in U.S. Pat. No. 10,543,368, “Seizure Onset Classification and Stimulation Parameter Selection,” which is incorporated by reference. Examples of features extracted from EEG records include spectral power in delta, theta, alpha, beta, low gamma and high gamma bands. A list of other features is disclosed in U.S. Pat. No. 10,543,368.

With further regard to spread-seizure EEG records, the ATC model 606 may be further configured to classify a spread-seizure EEG record as a particular activity type based on the spread time (i.e., the difference in seizure onset time on lead 1 vs lead 2) for that EEG record. For example, a spread-seizure EEG record with a spread time above a threshold value, e.g., 10 seconds, and brain activity frequency above a certain threshold, e.g., 50 Hz, may be classified by the ATC model 606 as a low voltage fast activity pattern. As another example, if the spread time is below 5 seconds and the brain activity frequency is below 20 Hz, the ATC may classify it as theta onset pattern.

With reference to FIG. 7A, the ATC model 606 may classify an EEG record of a local seizure on lead 1 as containing an activity type 1, such as semi-rhythmic beta type activity. An example of such an EEG record is shown in panel (e) of FIG. 7A.

With reference to FIG. 7B, the ATC model 606 may classify an EEG record of a local seizure on lead 2 as containing an activity type 2, such as high amplitude beta activity. An example of such an EEG record is shown in panel (a) of FIG. 7B.

With reference to FIG. 7C, the ATC model 606 may classify EEG record classified of a spread seizure on lead 1 and lead 2 as containing an activity type 3, such as rhythmic theta. An example of such an EEG record is shown in panel (b) of FIG. 7C.

Returning to FIG. 6, the therapy suggestion module 608 receives information resulting from the operations of the ESC model 602, the SSC model 604, and the ATC model 606, and processes the information to provide therapy suggestions. The information includes seizure spread pattern type information determined by the SSC model 604, seizure activity type information determined by the ATC model 606, and seizure information, e.g., no seizure, determined by the ESC model 602. Further disclosure of the operation of therapy suggestion module 608 is provided below.

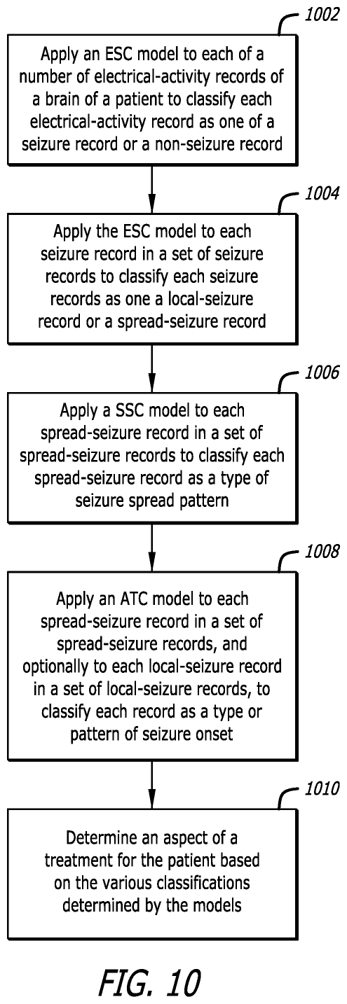

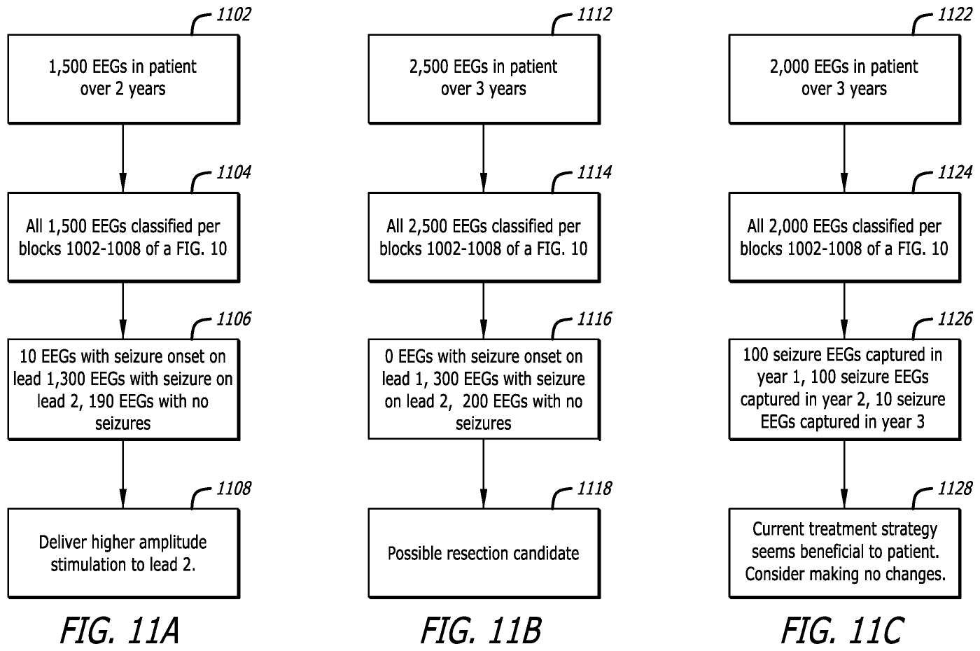

FIG. 10A is a flowchart of a method of assessing electrical activity of the brain. The method may be performed by the records classification processor 104 disclosed herein.

At block 1002, an ESC model 602 is applied to each of a number of electrical-activity records of a brain of a patient, each of which are sensed by a corresponding one of a plurality of sensing channels of an implanted medical device. The ESC model 602 is configured to classify electrical-activity records as one of a seizure record or a non-seizure record.

At block 1004, the ESC model 602 is further applied to each seizure record in a set of seizure records. Each seizure record comprises a first seizure record captured by a first channel of the plurality of sensing channels and a second seizure record captured by a second channel of the plurality of sensing channels. The ESC model 602 is configured to classify seizure records as one of a local-seizure record or a spread-seizure record.

At block 1006, a SSC model 604 is applied to each spread-seizure record in a set of spread-seizure records. The SSC model 604 is configured to classify the spread-seizure records as a type of seizure spread pattern.

At block 1008, an ATC model 606 is applied to each spread-seizure record in the set of spread-seizure records. The ATC model 606 may also be applied to each local-seizure record in a set of local-seizure records. The ATC model 606 is configured to classify the spread-seizure records and local-seizure record as a type or pattern of seizure onset.

At block 1010, an aspect of a treatment for the patient is determined based on the various classifications determined by the models.

In some embodiments, a treatment module 608 may be configured to determine an aspect of a treatment based on the type of seizure spread pattern for each spread-seizure record in the set of spread-seizure records. For example, the treatment module 608 may determine site stimulation for delivery of stimulation therapy in the form of electrical pulses. To this end, the treatment module 608 may determine a metric for each type of seizure spread pattern in the set of spread-seizure records. The metric may be a count of the number of occurrences of each type of seizure spread pattern over a period of time. The treatment module 608 processes the respective counts to determine a dominate type of seizure spread pattern, and selects a stimulation site based on the dominate type of seizure spread pattern. The treatment module 608 may apply logic to the counts and suggest a stimulation site as follows: for a dominate first-channel-to-second-channel seizure spread pattern, the treatment module 608 may suggest a stimulation site corresponding to a first location of the brain (where the first channel electrodes are located); for a dominate second-channel-to-first-channel seizure spread pattern, the treatment module 608 may suggest a stimulation site corresponding to a second location of the brain (where the second channel electrodes are located); and for a dominate non-spread seizure spread pattern, the treatment module 608 may suggest a stimulation site corresponding to one or both of the first location of the brain and the second location of the brain.

The treatment module 608 may be configured to determine an aspect of treatment based on the type of seizure spread pattern and a time of seizure spread for each spread-seizure record in the set of spread-seizure records. For example, the treatment module 608 may determine one or both of a stimulation site and a location for a surgical resection of the brain. To this end, the treatment module 608 may determine a metric set for each type of seizure spread pattern in the set of spread-seizure records. The metric set may include a count of the number of occurrences of each type of seizure spread pattern over a period of time, and a measure of time of seizure spread for the type of seizure spread. The measure of time of seizure spread may be a statistical measure, e.g., mean, median, mode, of the individual times of seizure spread for each type of seizure spread pattern. The treatment module 608 processes the respective metric sets to determine a dominate type of seizure spread pattern. Having determined the dominate type of seizure spread pattern, the treatment module 608 selects a stimulation site and/or a location for surgical resection based on the dominate type of seizure spread pattern and the measure of time of seizure spread for the dominate type of seizure spread.

The treatment module 608 may apply logic to the dominate type of seizure spread pattern and its corresponding measure of time of seizure spread and suggest a stimulation site as follows: For a dominate first-channel-to-second-channel seizure spread pattern, the treatment module 608 may suggest: a) a stimulation site corresponding to only a first location of the brain when the measure of time of seizure spread is at or above a threshold duration (e.g., greater than 10 seconds), orb) a stimulation site corresponding to both the first location of the brain and a second location of the brain when the measure of time of seizure spread is at or below a threshold duration (e.g., less than 10 seconds). For a dominate second-channel-to-first-channel seizure spread pattern, the treatment module 608 may suggest: a) a stimulation site corresponding to only the second location of the brain when the measure of time of seizure spread is at or above a threshold duration (e.g., greater than 10 seconds), or b) a stimulation site corresponding to both the first location of the brain and the second location of the brain when the measure of time of seizure spread is at or below a threshold duration (e.g., less than 10 seconds).

The treatment module 608 may apply logic to the dominate type of seizure spread pattern and its corresponding measure of time of seizure spread and suggest a location of the brain for surgical resection as follows: For a dominate first-channel-to-second-channel seizure spread pattern, the treatment module 608 may suggest: a) the location corresponding to a first location of the brain when the measure of time of seizure spread is at or above a threshold duration (e.g., greater than 5 seconds), and b) the location corresponding to no location of the brain when the measure of time of seizure spread is at or below a threshold duration (e.g., less than 5 seconds). In the case of no location, the treatment module 608 is suggesting that the patient not undergo a resection surgery. For a dominate second-channel-to-first-channel seizure spread pattern, the treatment module 608 may suggest: a) the location corresponding to a second location of the brain when the measure of time of seizure spread is at or above a threshold duration (e.g., greater than 5 seconds), and b) the location corresponding to no location of the brain when the measure of time of seizure spread is at or below a threshold duration (e.g., less than 5 seconds).

In some embodiments, the treatment module 608 may be configured to determine an aspect of treatment based on the type of seizure spread pattern, a seizure onset type for the first seizure record, and a seizure onset type for the second seizure record for each spread-seizure record in the set of spread-seizure records. For example, the treatment module 608 may determine a stimulation parameter for delivering electrical stimulation therapy. To this end, the treatment module 608 may derive a metric set for each type of seizure spread pattern in the set of spread-seizure records. The metric set may include a count of the number of occurrences of each type of seizure spread pattern over a period of time, and a count of each respective seizure onset type. The treatment module 608 processes the respective metric sets to determine a dominate type of seizure spread pattern and a dominate seizure onset type, and selects the stimulation parameter based on the dominate type of seizure spread pattern and the dominate seizure onset type. The treatment module 608 may apply logic to a dominate type of seizure spread pattern and a dominate seizure onset type and suggest a stimulation parameter as follows: For example, if the seizure spread pattern is from lead 1 to lead 2, and the seizure onset type is low voltage fast type onset, the treatment module may perform brain stimulation at 200 Hz, 1 μC. On the other hand, if the seizure spread pattern is from lead 2 to lead 1, and the seizure onset type is theta onset, the treatment module may perform brain stimulation at 7 Hz, 3 μC.

In this embodiment, an ATC model 606 is applied to each of the first seizure record and the second seizure record to determine the seizure onset type for the first seizure record and the seizure onset type for the second seizure record. The seizure onset type may be one of low voltage fast, hypersynchronous, attenuation, multiple, rhythmic delta, rhythmic theta, rhythmic alpha, semi-rhythmic beta.

With reference to 11A, in one example operation of the method of FIG. 10 a suggested stimulation parameter is provided by the treatment module 608. To this end, at block 1102, a dataset of 1500 EEG records for a patient is accessed by the records classification processor 104. The EEG records were collected over a two year period. At block 1104, the 1500 EEG records are processed in accordance with one or more of blocks 1002, 1004, 1006, and 1008 of FIG. 10. At block 1106, the classifications determined by one or more of the ESC model 602, the SSC model 604, and the ATC model 606 for the 1500 EEG records are provided to the treatment module 608, which determines the following metrics:

1) 10 EEG records classified as a local seizure on lead 1

2) 1,300 EEG records classified as a local seizure on lead 2

3) 190 EEG records classified as a non-seizure.

Based on these metrics, the treatment module 608 determines that a stimulation parameter used by the implanted medical device to delivery stimulation therapy may benefit the patient. In this example, the suggestion is to increase the amplitude of stimulation delivered through lead 2.

With reference to 11B, in another example operation of the method of FIG. 10 a suggested surgical procedure is provided treatment module 608. To this end, at block 1112, a dataset of 2500 EEG records for a patient is accessed by the records classification processor 104. The EEG records were collected over a three year period. At block 1114, the 2500 EEG records are processed in accordance with one or more of blocks 1002, 1004, 1006, and 1008 of FIG. 10. At block 1116, the classifications determined by one or more of the ESC model 602, the SSC model 604, and the ATC model 606 for the 2500 EEG records are provided to the treatment module 608, which determines the following metrics:

1) 0 EEG records classified as a local seizure on lead 1

2) 300 EEG records classified as a local seizure on lead 2

3) 2200 EEG records classified as non-seizures.

Based on these metrics, the treatment module 608 determines that a surgical procedure may benefit the patient. In this example, the suggestion is to perform a resection on the patient's brain.

With reference to 11C, in another example operation of the method of FIG. 10 a suggested to maintain current treatment is provided treatment module 608. To this end, at block 1122, a dataset of 2000 EEG records for a patient is accessed by the records classification processor 104. The EEG records were collected over a three year period. At block 1104, the 2000 EEG records are processed in accordance with one or more of blocks 1002, 1004, 1006, and 1008 of FIG. 10. At block 1106, the classifications determined by one or more of the ESC model 602, the SSC model 604, and the ATC model 606 for the 2000 EEG records are provided to the treatment module 608, which determines the following metrics:

1) 100 EEG records classified as seizures in first year

2) 100 EEG records classified as seizures in second year

3) 10 EEG records classified as seizures in third year

Based on these metrics, the treatment module 608 determines that the current treatment is beneficial since the number of seizures has reduced. In this example, the suggestion is to maintain the current treatment.

As mentioned above, therapy suggestions determined by the treatment module 608 may also be based on activity types/patterns classification of seizure EEG records, seizure spread patterns, time of spread classification in spread-seizure EEG records, or a combination of these classification factors. For example, in patient where 95% of seizures are classified as local seizures on lead 1, and only 5% of seizures are classified as local seizures on lead 2, and if the majority of local seizures on lead 1 are classified by the ATC model a low voltage fast type, the treatment module 608 may suggest to increase the stimulation amplitude on lead 1, and to stimulate at a frequency that is effective for treating low voltage fast type seizures, such as 250 Hz.

In another example, if most of the spread-seizure EEG records are classified by the SSC module as having a lead2-to-lead1 seizure spread pattern and a slow spread (˜20 seconds), and classified by the ATC model 606 as high amplitude beta type onset on lead 2, then the treatment module 608 may suggest to stimulate lead 2 with high amplitude stimulation at a pulse width that is effective in treating high amplitude best type onset, such as 100 μs.

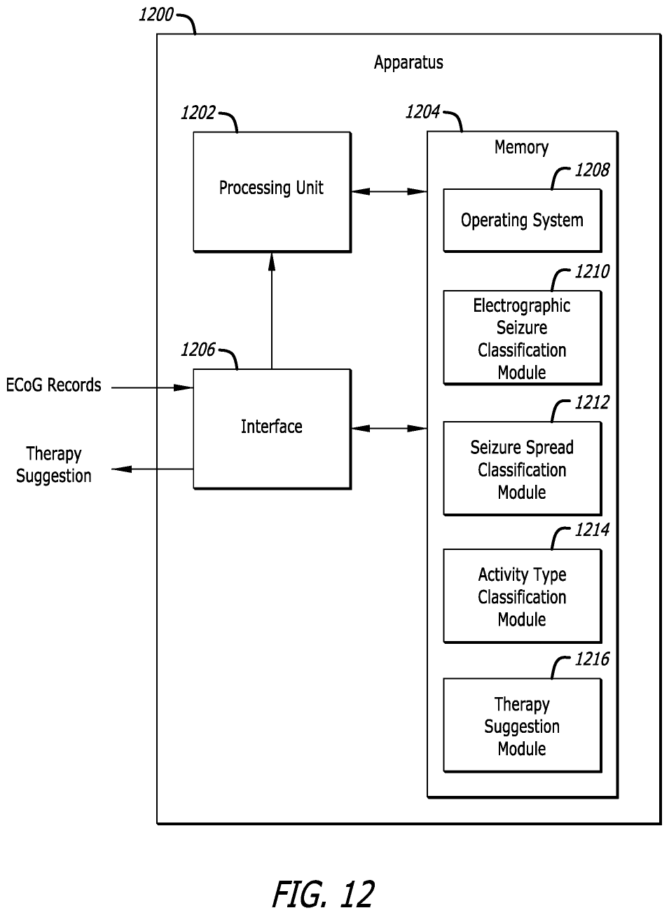

FIG. 12 is a schematic block diagram of an apparatus 1200 corresponding to the records classification processor 104 of FIG. 1. The apparatus 1200 is configured to execute instructions related to the records classification processes described above with reference to FIGS. 6, 7, 8A, and 8B. The apparatus 1200 may be embodied in any number of processor-driven devices, including, but not limited to, a server computer, a personal computer, one or more networked computing devices, an application-specific circuit, a minicomputer, a microcontroller, and/or any other processor-based device and/or combination of devices.

The apparatus 1200 may include one or more processing units 1202 configured to access and execute computer-executable instructions stored in at least one memory 1204. The processing unit 1202 may be implemented as appropriate in hardware, software, firmware, or combinations thereof. Software or firmware implementations of the processing unit 1202 may include computer-executable or machine-executable instructions written in any suitable programming language to perform the various functions described herein. The processing unit 1202 may include, without limitation, a central processing unit (CPU), a digital signal processor (DSP), a reduced instruction set computer (RISC) processor, a complex instruction set computer (CISC) processor, a microprocessor, a microcontroller, a field programmable gate array (FPGA), a System-on-a-Chip (SOC), or any combination thereof. The apparatus 1200 may also include a chipset (not shown) for controlling communications between the processing unit 1202 and one or more of the other components of the apparatus. The processing unit 1202 may also include one or more application-specific integrated circuits (ASICs) or application-specific standard products (ASSPs) for handling specific data processing functions or tasks.

The memory 1204 may include, but is not limited to, random access memory (RAM), flash RAM, magnetic media storage, optical media storage, and so forth. The memory 1204 may include volatile memory configured to store information when supplied with power and/or non-volatile memory configured to store information even when not supplied with power. The memory 1204 may store various program modules, application programs, and so forth that may include computer-executable instructions that upon execution by the processing unit 1202 may cause various operations to be performed. The memory 1204 may further store a variety of data manipulated and/or generated during execution of computer-executable instructions by the processing unit 1202.

The apparatus 1200 may further include one or more interfaces 1206 that may facilitate communication between the apparatus and one or more other apparatuses. For example, the interface 1206 may be configured to receive EEG records from a database and to output information, e.g., therapy suggestions, to a display. Communication may be implemented using any suitable structure or communications standard. For example, communication with a database may be through a LAN interface that implement protocols and/or algorithms that comply with various communication standards of the Institute of Electrical and Electronics Engineers (IEEE), such as IEEE 802.11. Communication with a user interface and display may be through wired structures.

The memory 1204 may store various program modules, application programs, and so forth that may include computer-executable instructions that upon execution by the processing unit 1202 may cause various operations to be performed. For example, the memory 1204 may include an operating system module (O/S) 1208 that may be configured to manage hardware resources such as the interface 1206 and provide various services to applications executing on the apparatus 1200.

The memory 1204 stores additional program modules such as: (1) an electrographic seizure classification module 1210 that receives and process EEG records to classify or categorize the records as a spread seizure, a local seizure, or a non-seizure; (2) a seizure spread classification module 1212 that receives EEG records of spread seizures and further processes them to predict times of seizure onset and to further categorize the spread seizure accordingly; (3) an activity type classification module 1214 that receives EEG records of spread seizures and local seizures and further processes the records to determine a seizure onset type; and (4) a therapy suggestion module 1216 that determines and outputs therapy suggestions based on the outputs of the seizure onset classification module and the activity type classification module. Each of these modules includes computer-executable instructions that when executed by the processing unit 1202 cause various operations to be performed, such as the operations described immediately above and earlier with reference to FIG. 3.

The apparatus 1200 and modules disclosed herein may be implemented in hardware or software that is executed on a hardware platform. The hardware or hardware platform may be a general purpose processor, a digital signal processor (DSP), an application specific integrated circuit (ASIC), a field programmable gate array (FPGA) or other programmable logic component, discrete gate or transistor logic, discrete hardware components, or any combination thereof, or any other suitable component designed to perform the functions described herein. A general-purpose processor may be a microprocessor, but in the alternative, the processor may be any conventional processor, controller, microcontroller, or state machine. A processor may also be implemented as a combination of computing components, e.g., a combination of a DSP and a microprocessor, a plurality of microprocessors, one or more microprocessors in conjunction with a DSP, or any other such configuration.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. A method of assessing electrical activity of a brain, the method comprising:
for each of a plurality of electrical-activity records of a brain, applying a machine-learned electrographic seizure classification (ESC) model to the electrical-activity record to classify the electrical-activity record as one of a seizure record or a non-seizure record, wherein each of the plurality of electrical-activity records is sensed by a corresponding one of a plurality of sensing channels of an implanted medical device;
for each seizure record in a set of seizure records, applying the machine-learned ESC model to the seizure record to classify the seizure record as one of a local-seizure record or a spread-seizure record, wherein the seizure record comprises a first seizure record captured by a first channel of the plurality of sensing channels and a second seizure record captured by a second channel of the plurality of sensing channels; and
for each spread-seizure record in a set of spread-seizure records, applying a machine-learned seizure spread classification (SSC) model to the spread-seizure record to classify the spread-seizure record as a type of seizure spread pattern.

2. The method of claim 1, wherein a seizure record is classified as a local-seizure record when the ESC model determines a seizure is present in only one of the first seizure record and the second seizure record.

3. The method of claim 1, wherein a seizure record is classified as a spread-seizure record when the ESC model determines a seizure is present in each of the first seizure record and the second seizure record.

4. The method of claim 1, wherein:
the first channel is defined by one or more electrodes implanted at a first location of the brain and the second channel is defined by one or more electrodes implanted at a second location of the brain, and
the spread-seizure record is classified as:
a first-channel-to-second-channel seizure spread pattern when the SSC model determines a time of seizure onset in the first seizure record precedes a time of seizure onset in the second seizure record by a threshold duration,
a second-channel-to-first-channel seizure spread pattern when the SSC model determines a time of seizure onset in the second seizure record precedes a time of seizure onset in the first seizure record by a threshold duration, and
a non-spread seizure spread pattern when the SSC model determines a duration between a time of seizure onset in the first seizure record and a time of seizure onset in the second seizure record is within a threshold range.

5. The method of claim 4, wherein the first location of the brain and the second location of the brain are on or within a same hemisphere of the brain.

6. The method of claim 4, wherein the first location of the brain and the second location of the brain are on or within opposite hemispheres of the brain.

7. The method of claim 1, further comprising:
determining an aspect of a treatment based on the type of seizure spread pattern for each spread-seizure record in the set of spread-seizure records.

8. The method of claim 7, wherein the aspect of a treatment corresponds to a stimulation site, and determining the stimulation site comprises:
for each type of seizure spread pattern included in the set of spread-seizure records, deriving a metric based a count of the type of seizure spread pattern;
processing the respective counts to determine a dominate type of seizure spread pattern; and
select a stimulation site based on the dominate type of seizure spread pattern.

9. The method of claim 8, wherein:
for a dominate first-channel-to-second-channel seizure spread pattern, the stimulation site corresponds to a first location of the brain,
for a dominate second-channel-to-first-channel seizure spread pattern, the stimulation site corresponds to a second location of the brain, and
for a dominate non-spread seizure spread pattern, the stimulation site corresponds to one or both of the first location of the brain and the second location of the brain.

10. The method of claim 1, further comprising:
determining an aspect of treatment based on the type of seizure spread pattern and a time of seizure spread for each spread-seizure record in the set of spread-seizure records.

11. The method of claim 10, wherein the aspect of a treatment corresponds to at least one of a stimulation site and a location of a surgical resection, and determining the stimulation site comprises:
for each type of seizure spread pattern included in the set of spread-seizure records, deriving a metric set comprising a count of the type of seizure spread pattern and a measure of time of seizure spread for the type of seizure spread;
processing the respective metric sets to determine a dominate type of seizure spread pattern; and
selecting at least one of a stimulation site and a location of a surgical resection based on the dominate type of seizure spread pattern and the measure of time of seizure spread for the dominate type of seizure spread.

12. The method of claim 11, wherein:
for a dominate first-channel-to-second-channel seizure spread pattern:
the stimulation site corresponds to only a first location of the brain when the measure of time of seizure is at or above a threshold duration, and
the stimulation site corresponds to both the first location of the brain and a second location of the brain when the measure of time of seizure is at or below a threshold duration; and
for a dominate second-channel-to-first-channel seizure spread pattern:
the stimulation site corresponds to only the second location of the brain when the measure of time of seizure is at or above a threshold duration, and
the stimulation site corresponds to both the first location of the brain and the second location of the brain when the measure of time of seizure at or below a threshold duration.

13. The method of claim 11, wherein:
for a dominate first-channel-to-second-channel seizure spread pattern:
the location corresponds to a first location of the brain when the measure of time of seizure is at or above a threshold duration, and
the location corresponds to no location of the brain when the measure of time of seizure is at or below a threshold duration; and
for a dominate second-channel-to-first-channel seizure spread pattern:
the location corresponds to a second location of the brain when the measure of time of seizure is at or above a threshold duration, and
the location corresponds to no location of the brain when the measure of time of seizure is at or below a threshold duration.

14. The method of claim 1, further comprising:
determining an aspect of treatment based on the type of seizure spread pattern, a seizure onset type for the first seizure record, and a seizure onset type for the second seizure record for each spread-seizure record in the set of spread-seizure records.

15. The method of claim 14, further comprising:
applying a machine-learned activity type classification (ATC) model to each of the first seizure record and the second seizure record to determine the seizure onset type for the first seizure record and the seizure onset type for the second seizure record.

16. The method of claim 15, wherein the aspect of the treatment corresponds to a stimulation parameter, and determining the stimulation parameter comprises:
for each type of seizure spread pattern included in the set of spread-seizure records, deriving a metric set comprising a count of the type of seizure spread pattern, and a count of each respective seizure onset type;
processing the respective metric sets to determine a dominate type of seizure spread pattern and a dominate seizure onset type; and
selecting the stimulation parameter based on the dominate type of seizure spread pattern and the a dominate seizure onset type.

17. The method of claim 16, wherein the seizure onset type corresponds to one of low voltage fast, hypersynchronous, attenuation, multiple, rhythmic delta, rhythmic theta, rhythmic alpha, semi-rhythmic beta.

18. An apparatus for assessing electrical activity of a brain, the apparatus comprising:
a memory having one or more machine-learned models, and
a processor couple to the memory and configured to:
for each of a plurality of electrical-activity records of a brain, applying a machine-learned electrographic seizure classification (ESC) model to the electrical-activity record to classify the electrical-activity record as one of a seizure record or a non-seizure record, wherein each of the plurality of electrical-activity records is sensed by a corresponding one of a plurality of sensing channels of an implanted medical device;
for each seizure record in a set of seizure records, applying the machine-learned electrographic seizure classification (ESC) model to the seizure record to classify the seizure record as one of a local-seizure record or a spread-seizure record, wherein the seizure record comprises a first seizure record captured by a first channel of the plurality of sensing channels and a second seizure record captured by a second channel of the plurality of sensing channels; and
for each spread-seizure record in a set of spread-seizure records, applying a machine-learned seizure spread classification (SSC) model to the spread-seizure record to classify the spread-seizure record as a type of seizure spread pattern.

19. The apparatus of claim 18, wherein the ESC model is configured to classify a seizure record as a local-seizure record when a seizure is present in only one of the first seizure record and the second seizure record.

20. The apparatus of claim 18, wherein the ESC model is configured to classify a seizure record as a spread-seizure record when a seizure is present in each of the first seizure record and the second seizure record.

21. The apparatus of claim 18, wherein:
the first channel is defined by one or more electrodes implanted at a first location of the brain and the second channel is defined by one or more electrodes implanted at a second location of the brain, and
the SSC model is configured to:
classify a spread-seizure record as a first-channel-to-second-channel seizure spread pattern when the SSC model determines a time of seizure onset in the first seizure record precedes a time of seizure onset in the second seizure record by a threshold duration, classify a spread-seizure record as a second-channel-to-first-channel seizure spread pattern when the SSC model determines a time of seizure onset in the second seizure record precedes a time of seizure onset in the first seizure record by a threshold duration, and
classify a spread-seizure record as a non-spread seizure spread pattern when the SSC model determines a duration between a time of seizure onset in the first seizure record and a time of seizure onset in the second seizure record is within a threshold range.

22. The apparatus of claim 21, further comprising a treatment module configured to:
derive a metric based a count of the type of seizure spread pattern for each type of seizure spread pattern included in the set of spread-seizure records;
process the respective counts to determine a dominate type of seizure spread pattern; and
select a stimulation site for delivery of stimulation therapy based on the dominate type of seizure spread pattern.

23. The apparatus of claim 21, further comprising a treatment module configured to:
derive a metric set comprising a count of the type of seizure spread pattern and a measure of time of seizure spread for the type of seizure spread, for each type of seizure spread pattern included in the set of spread-seizure records;
process the respective metric sets to determine a dominate type of seizure spread pattern; and
select at least one of stimulation site and a location for a resection surgery based on the dominate type of seizure spread pattern and the measure of time of seizure spread for the dominate type of seizure spread.

24. The apparatus of claim 21, further comprising a treatment module configured to:
derive a metric set comprising a count of the type of seizure spread pattern, and a count of each respective seizure onset type for each type of seizure spread pattern included in the set of spread-seizure records;
process the respective metric sets to determine a dominate type of seizure spread pattern and a dominate seizure onset type; and
select a stimulation parameter based on the dominate type of seizure spread pattern and the a dominate seizure onset type.

25. The apparatus of claim 24, further comprising:
a machine-learned activity type classification (ATC) model configured to be applied to each of the first seizure record and the second seizure record to determine a seizure onset type for the first seizure record and a seizure onset type for the second seizure record.
